(19) United States
(12) Patent Application Publication
(10) Pub. No.: US 2006/0275786 A1
(43) Pub. Date: Dec. 7, 2006

(54) Electrochemical detection of DNA binding

(76) Inventors: Yi-Tao Long, Saskatoon (CA); Chen-Zhong Li, Saskatoon (CA); Heinz-Bernhard Kraatz, Saskatoon (CA); Jeremy S. Lee, Saskatoon (CA)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/317,671

(22) Filed: Dec. 22, 2005

Related U.S. Application Data
(63) Continuation of application No. PCT/CA04/00957, filed on Jun. 25, 2004.
(63) Continuation-in-part of application No. 10/547,862, filed on Feb. 2, 2006.
(60) Provisional application No. 60/482,766, filed on Jun. 27, 2003.
(60) Provisional application No. 60/451,627, filed on Mar. 5, 2003.

(51) Int. Cl. C12Q 1/68; C12M 1/34

(52) U.S. Cl. 435/6; 435/287.2; 205/777

(57) ABSTRACT

In one aspect, the invention provides methods and apparatus for detecting a protein binding to a nucleic acid by measuring the impedence of a nucleic acid layer on an electrode, for example by AC impedance spectroscopy. In one embodiment, such methods may for example be used to detect a mismatch in a nucleic acid duplex.

### FIELD OF THE INVENTION

The invention is in the field of conductive nucleic acids, and electrochemical techniques for analysis of nucleic acids.

### BACKGROUND OF THE INVENTION

The electronic conductivity of DNA may be utilized in the development of DNA biosensors, so called “DNA chips” (Bixon et al., 1999; Schena et al., 1996; Fodor et al., 1993). One form of DNA chip consists of single-stranded DNA probes attached to a surface in an array format. The target DNA may be labelled with a fluorescent tag and successful hybridization to an individual probe may be detected fluorometrically. Electrochemical detection, on the other hand, may allow a direct readout of the signal (Takagi, 2001; Kelly et al., 1999). Electrochemical techniques include potential step chronoamperometry, dc cyclic voltammetry, and electrochemical impedance spectroscopy (Bard and Faulkner, 2001). Electrochemical DNA sensors may utilize electrochemically active DNA binding drugs such as the metal coordination complex Ru(bpy)₃²⁺ (Carter and Bard, 1987; Millan et al., 1994), electroactive dyes (Hashimoto et al., 1994), quinones (Kertesz et al., 2000; Ambroise and Maiya, 2000), and methyl blue (Tani et al., 2001; Kelley et al., 1997) as the detection markers. In other cases the simple redox probe, Fe(CN)₆^{3-/4-}, has been used in solution (Patolsky et al., 2001). In some of these techniques, target DNA need not be labeled in advance.

The electronic characteristics of surface modified electrodes can be probed with impedance spectroscopy and the data modeled by an equivalent circuit (Macdonald, 1987). Alternative methods of electrochemical impedance spectroscopy are for example disclosed in U.S. Pat. No. 6,556,001 (incorporated herein by reference). Electron transfer through self-assembled alkanethiol monolayers on metal surfaces has been intensively studied in recent years (Ulman, 1996). The impedance of an electrode undergoing heterogeneous electron transfer through a self-assembled monolayer is usually described on the basis of the model developed by Randles (Randles, 1947).

Duplex DNA contains a stacked π system and the conductivity of native DNA (B-DNA) has been hotly debated. Recent direct measurements suggest that B-DNA is a semiconductor with a wide band gap (Storm et al., 2001); (Rakitin et al., 2001); (Porath et al., 2000); (Murphy et al., 1993). The conductivity of DNA can be improved by deposition of silver atoms along its length but the process is essentially irreversible (Braun et al., 1998). Another possibility is to convert B-DNA to M-DNA by the addition of divalent metal ions (Zn²⁺, Co²⁺ and Ni²⁺) at pHs above 8.5 (Lee et al., 1993) (Aich et al., 1999). In M-DNA, it is proposed that the metal ions replace the imino protons of guanine and thymine in every base pair but the structure can be converted back to B-DNA by chelating the metal ions with EDTA or reducing the pH. Electron transport through M-DNA can be monitored by fluorescence spectroscopy of duplexes labelled at opposite ends with donor and acceptor chromophores. Upon formation of M-DNA the donor is quenched but only when the acceptor is on the same DNA molecule (Aich et al., 1999; Aich et al., 2002). Recent direct measurements have confirmed that M-DNA shows metallic-like conductivity and electron transfer can be observed in duplexes as long as 500 base pairs (Rakitin et al., 2001). Therefore, M-DNA may be useful in biosensor applications by allowing a direct electronic readout of the state of the DNA.

### SUMMARY OF THE INVENTION

In one aspect, the invention provides hardware and software for an impedance spectroscopy system that characterizes polymers such as nucleic acids by measuring impedance at various frequencies. The hardware may for example provide voltage and current inputs to a sample at various frequencies and measure the resulting impedance. The software may store equivalent circuit parameters for multiple samples, control the hardware inputs to the sample, display measurement data, display results, and notify an operator if results exceed preset limits.

In one aspect, the invention provides methods for diluting nucleic acid monolayers on an electrode, to facilitate electrochemical analysis of protein binding by the monolayer. In one embodiment of this aspect of the invention, a DNA binding protein is used that recognizes mismatches in a tethered dsDNA. It is demonstrated that the use of a nucleotide mismatch binding protein in this way enhances the sensitivity of impedance spectroscopy so as to facilitate the detection of single base pair mismatches in a nucleic acid duplex. Accordingly, this aspect of the invention provides a system that may for example be used to detect single nucleotide polymorphisms. An electrode may for example be coated with a single strand of a selected nucleic acid sequence. The electrode coated electrode may then be hybridized to a population of test nucleic acids, to for a double stranded nucleic acid coated electrode. The electrode may then be exposed to a nucleic acid binding protein, such as a mismatch binding protein, and the nucleic acid monolayer may then be analyzed by electrochemical means, such as electrochemical impedance spectroscopy (EIS).

In various aspects, the invention provides methods for detecting binding of a moiety to a nucleic acid tethered to an electrode in an electrochemical circuit. A plurality of nucleic acids may for example form a monolayer of nucleic acid duplexes on the electrode. The nucleic acids may be comprised of naturally occurring monomers, such as DNA and RNA, or may have synthetic substituents comprised of a wide range of alternative monomeric units.

Methods of the invention may include the steps of: a) applying electrical energy to the electrode in the electrochemical circuit; b) collecting electrochemical circuit data related to the impedance of the nucleic acid on the electrode in the circuit; and, c) fitting the electrochemical circuit data to a circuit model to obtain circuit performance information indicative of binding of a moiety to the nucleic acid, such as a nucleic acid duplex.

In alternative aspects, the invention provides systems for detecting binding of a moiety to a nucleic acid. Such systems may for example include: a) means such as an electrical current source for applying electrical energy to the electrode in the electrochemical circuit; b) means such as a controller for collecting electrochemical circuit data related to the impedance of the nucleic acid duplex on the electrode in the circuit; and, c) means such as an analyzer for fitting the electrochemical circuit data to a circuit model to obtain circuit performance information indicative of binding of the moiety to the nucleic acid, such as a nucleic acid duplex. Such systems may further comprise a display or means for displaying the circuit performance information; and/or a recorder or means for recording the circuit performance information. The circuit performance information may for example be plotted on a Nyquist plot.

In alternative embodiments, collecting electrochemical circuit data may include measuring impedance spectra, such as impedance spectra measured in the frequency domain. Various electrochemical circuit parameters provide data that is related to the impedance of the nucleic acid duplex. For example, the real and imaginary impedance of a nucleic acid or monolayer is related to electrochemical parameters such as the Warburg impedance, the capacitance of the monolayer, the charge transfer resistance and the rate of electron transfer. Such parameters may also be used to distinguish bound from unbound DNA in a sample.

The electrochemical circuit data of the invention may include a measure of complex impedance. In some embodiments, electrical energy may be applied in an impedance spectroscopy system, and the impedance spectroscopy system may involve applying a sinusoidal signal at a constant frequency and a constant amplitude within a discrete period. In selected embodiments, the circuit model may include circuit elements, such as:
a solution resistance Rs;
a charge transfer resistance RCT;
a constant-phase element CPE;
a mass transfer element W (Warburg impedance); and,
a resistance in parallel Rx;
wherein the circuit elements are arranged as illustrated in FIG. 1.

In some embodiments, the nucleic acid may be a deoxyribonucleic acid (DNA), and the nucleic acid duplex may be an double helix. In some embodiments, the nucleic acid may comprise M-DNA, a metal-containing nucleic acid duplex comprising a first strand of nucleic acid and a second strand of nucleic acid, the first and the second nucleic acid strands comprising a plurality of nitrogen-containing aromatic bases covalently linked by a backbone, the nitrogen-containing aromatic bases of the first nucleic acid strand being joined by hydrogen bonding to the nitrogen-containing aromatic bases of the second nucleic acid strand, the nitrogen-containing aromatic bases on the first and the second nucleic acid strands forming hydrogen-bonded base pairs in stacked arrangement along the length of the conductive metal-containing nucleic acid duplex, the hydrogen-bonded base pairs comprising an interchelated divalent metal cation coordinated to a nitrogen atom in one of the aromatic nitrogen-containing aromatic bases.

The electrochemical circuit may for example include an aqueous electrolyte and the nucleic acid may be tethered and solvated in the aqueous electrolyte. A redox probe may be provided in the aqueous solution.

### BRIEF DESCRIPTION OF THE DRAWINGS

FIG. 1 illustrates the equivalent circuit mode for B-DNA and M-DNA. The circuit within the dotted box is the standard Randles circuit. Rₛ: solution resistance, Rₓ: resistance through the DNA, R_{ct}: charge transfer resistance, CPE: constant phase element. W: Warburg impedance

FIG. 2 is a schematic illustration of native DNA (B-DNA) and metal DNA (M-DNA) on a gold electrode surface. As illustrated, the Zn²⁺ ions may be though of as binding to the outside of the M-DNA as well as being inserted into the helix.

FIG. 3 shows cyclic voltammograms for (a) bare gold and (b) 20 base pair duplex B-DNA assembled on gold electrode in 4 mM K₃[Fe(CN)₆]/K₄[Fe(CN)₆] (1:1), 20 mM NaClO₄ and 20 mM Tris-ClO₄ buffer solution (pH 8.6). Scan rate, 50 mV/s.

FIG. 4 shows XPS spectra of (a) bare gold, (b) 20 base pair duplex B-DNA assembled on gold and (c) 20 base pair duplex M-DNA assembled on gold.

FIG. 5 shows Nyquist plots (Zim vs Zre) with 4 mM Fe(CN)₆^{3-/4-} (1:1) mixture as redox probe 20 mM Tris-ClO₄ and 20 mM NaClO₄ solution, applied potential 0.250 V vs. Ag/AgCl. In all cases the measured data points are shown as ◯ with the calculated fit to the Randles circuit as ______ or modified Randles circuit as ______. (A) Bare gold electrode, (B) 20 base pair duplex B-DNA assembled on gold electrode, (C) 20 base pair duplex M-DNA assembled on gold electrode and (D) 20 base pair duplex B-DNA assembled on gold electrode with 0.4 mM Zn²⁺ at pH 7.0 (◯) or with 0.4 mM Mg²⁺ at pH 8.6 (Γ).

FIG. 6 shows Nyquist plots in the absence of a redox probe for (A) 20 base pair duplex B-DNA assembled on gold (Γ) and (B) 20 base pair duplex M-DNA assembled on gold (◯). The experimental data were fit to the equivalent circuit shown.

FIG. 7 shows Nyquist plots with Fe(CN)₆^{3-/4-} as redox probe for 15 base pair duplex monolayers as B-DNA (Γ) or M-DNA (◯), 20 base pair duplex monolayers as B-DNA (∘) or M-DNA (●), and 30 base pair duplex monolayers as B-DNA (Δ) or M-DNA (▴). The data points were fit to the modified Randles circuit as described in the text.

FIG. 8 shows Nyquist plot for the Impedance measurements for B-DNA and M-DNA modified gold electrode in 5 mM Ru(NH₃)^{3+/2+}, 20 mM Tris-ClO₄ buffer solution (pH, 8.6), applied potential −0.10V vs. Ag/AgCl.

FIG. 9 is a schematic showing DNA mismatches in duplexes attached to a surface, as discussed in Example 2.

FIG. 10 is a graph showing impedance spectra for a perfect duplex and one containing a middle mismatch under B-DNA and M-DNA conditions, as discussed in Example 2.

FIG. 11 is data showing the detection of a mismatch in a nucleic acid duplex on an electrode, using a DNA binding protein, as described in Example 3. The figure shows two Nyquist plots (Zᵢₘ vs Zᵣₑ) for the Faradaic impedence measurements in the presence of 5 mM [Fe(CN)₆]^{3-/4-} in 20 mM Tris-ClO₄ buffer (pH 8.5) containing 100 mM NaClO₄ for (A) perfectly matched ds-DNA modified electrode; (B) single base pair (AC) mismatched ds-DNA modified electrode. In both cases, (a) ds-DNA modified electrode, (b) the ds-DNA modified electrode treated with 1 mM butanethiol, (c) after further interaction with MutS protein.

### DETAILED DESCRIPTION OF THE INVENTION

In one aspect of the invention, impedance spectroscopy has been used to probe the electronic properties of B-and M-DNA self-assembled monolayers on gold electrodes.

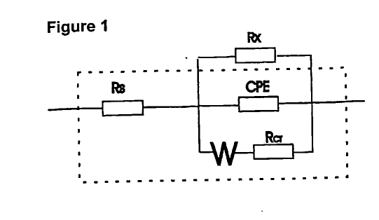

By way of background, FIG. 1 illustrates an electrical circuit modelling the impedance of an electrode undergoing heterogeneous electron transfer through a self-assembled monolayer, which may be described using a model developed by Randles (Randles, 1947). The equivalent electrical circuit (FIG. 1 in dotted box) consists of resistive and capacitance elements. Rₛ is the solution resistance, R_{ct} is the charge transfer resistance, C is the double-layer capacitance and W is the Warburg impedance due to mass transfer to the electrode. In general the Randles circuit provides a good model for the behaviour of alkanethiol monolayers. However, monolayers of HMB (4′-hydroxy-4-mercaptobiphenyl) which contain a conjugated π system are only described well by the Randles circuit if an additional resistance is added in parallel (Rₓ in FIG. 1) (Janek et al., 1998).

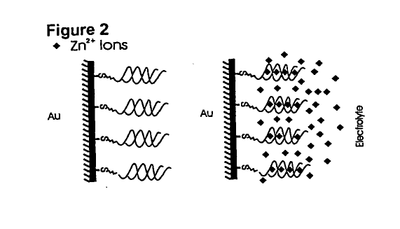

As shown in FIG. 2, upon addition of Zn²⁺ to form M-DNA the ions are inserted into the DNA helix as well as binding to the phosphate backbone outside the helix. The conversion of B- to M-DNA gives rise to characteristic changes in the impedance spectra which was observed for 15, 20 and 30 base pair duplexes. It was found that the modified Randles circuit which includes Rₓ, a resistance in parallel, may be used to give a good fit to the experimental data (FIG. 1). Under these conditions, M-DNA appears to decrease both Rₓ and R_{ct}, and promote electron transfer through the monolayer.

Various aspects of the invention involve M-DNA, a form of conductive metal-containing oligonucleotide duplex. In alternative aspects of the invention, the conductive metal-containing oligonucleotide duplex may include a first nucleic acid strand and a second nucleic acid strand, the first and second nucleic acid strands including respective pluralities of nitrogen-containing aromatic bases covalently linked by a backbone. The nitrogen-containing aromatic bases of the first nucleic acid strand may be joined by hydrogen bonding to the nitrogen-containing aromatic bases of the second nucleic acid strand. The nitrogen-containing aromatic bases on the first and the second nucleic acid strands may form hydrogen-bonded base pairs in stacked arrangement along a length of the conductive metal-containing oligonucleotide duplex. The hydrogen-bonded base pairs may include an interchelated metal cation coordinated to a nitrogen atom in one of the nitrogen-containing aromatic bases.

The interchelated metal cation may include an interchelated divalent metal cation. The divalent metal cation may be selected from the group consisting of zinc, cobalt and nickel. Alternatively, the metal cation may be selected from the group consisting of the cations of Li, Be, Na, Mg, Al, K, Ca, Sc, Ti, V, Cr, Mn, Fe, Co, Ni, Cu, Zn, Ga, Ge, As, Rb, Sr, Y, Zr, Nb, Mo, Tc, Ru, Rh, Pd, Ag, Cd, In, Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, TI, Pb, Bi, Po, Fr, Ra, Ac, Th, Pa, U, Np and Pu.

The first and the second nucleic acid strands may include deoxyribonucleic acid and the nitrogen-containing aromatic bases may be selected from the group consisting of adenine, thymine, guanine and cytosine. The divalent metal cations may be substituted for imine protons of the nitrogen-containing aromatic bases, and the nitrogen-containing aromatic bases may be selected from the group consisting of thymine and guanine. At least one of the nitrogen-containing aromatic bases may include thymine, having an N3 nitrogen atom, and the divalent metal cation may be coordinated by the N3 nitrogen atom. Alternatively, at least one of the nitrogen-containing aromatic bases may include guanine, having an N1 nitrogen atom, and the divalent metal cation may be coordinated by the N1 nitrogen atom.

In various aspects of the invention, as disclosed in the following examples, DNA monolayers may be assembled on a gold surface and assessed by cyclic voltammetery (CV) or X-ray photoelectron spectroscopy (XPS). As shown in the examples, the CV spectra may provide good evidence for a densely-packed monolaryer with good blocking against Fe(CN)₆^{3-/4-}, From the XPS, the film thickness may be estimated based on the exponential attenuation of the Au 4f signal, calculated in the examples to be 45 Å. (Pressprich et al., 1989). A 20 base pair duplex may be expected to have a length of about 70 Å so a measured thickness of 45 Å is for examples consistent with the DNA protruding from the surface at an angle of about 50°. In general duplex DNA attaches through the linker as compared to single-stranded DNA which can also attach through the bases (Heme and Tarlov, 1997). In the examples, the value of 162.4 eV for the S₂ₚ peak is in good agreement with previous reports for alkylthiols indicating that the DNA is interacting with the surface through a S—Au bond (Ishida et al., 1999).

AC impedance spectroscopy is a known method to probe and model the interfacial characterization of electrodes (Bard and Faulkner, 2001). Data may for example be presented as Nyquist plots (Zᵢₘ vs Zᵣₑ) in which characteristic changes may be readily observed and interpreted. The complex impedance may be presented as the sum of the real, Zᵣₑ (ω), and the imaginary, Zᵢₘ (ω) components that may originate mainly from the resistance and capacitance of the measured electrochemical system, respectively. As exemplified herein, the Nyquist plot for a bare electrode is a semicircle region lying on the Zᵣₑ axis followed by a straight line. The semicircle portion, measured at higher frequencies, putatively corresponds to direct electron transfer limited process, whereas the straight linear portion, observed at lower frequencies, putatively represents the diffusion controlled electron transfer process. The modification of the metallic surface with an organic layer may decrease the double layer capacitance and retard the interfacial electron transfer rates compared to a bare metal electrode (Finklea et al., 1993; Kharitonov et al., 2000).

In alternative embodiments, electrochemical techniques other than impedance spectroscopy may be utilized. For example, AC voltammetry, cyclic voltammetry and chronoamperometry (particulalry if a redox-active protein is used as the nucleic acid binding moiety), may be used to measure an electrochemical property of a nucleic acid monolayer on an electrode. In some embodiments, nucleic acid monolayers may be diluted to provide nucleic acid densities on an electrode surface that are optimized for analysis of moiety binding by a selected protocol.

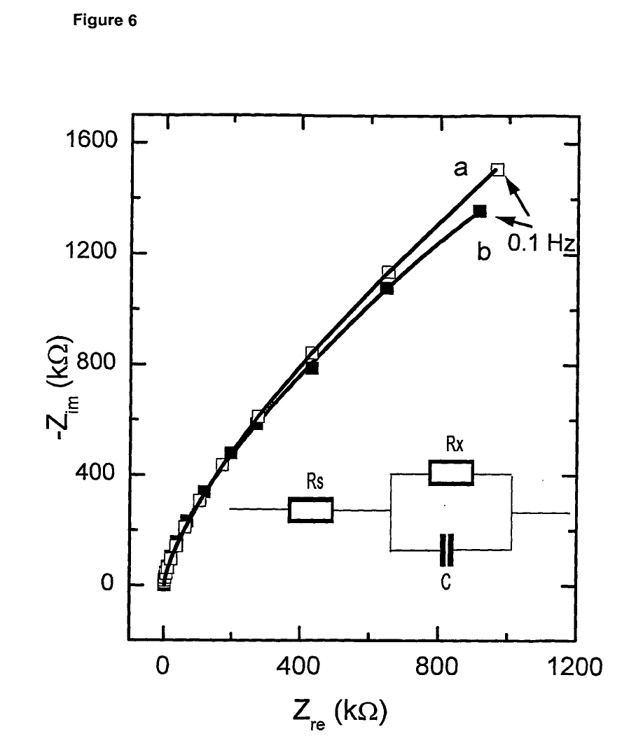

In some embodiments, data analysis may require modeling the electrode kinetics with an equivalent circuit consisting of electrical components. For many monolayers the commonly accepted equivalent circuit is based on the Randles model, as shown in FIG. 1. However, in order to obtain a good fit to the data from the examples disclosed herein, a parallel interfacial resistance Rₓ was added to the equivalent circuit, nominally corresponding to electron transfer through the DNA. Evidence for a parallel interfacial resistance may for example be provided by impedance measurements without the Fe(CN)₆^{3-/4-}, redox-active probe (see FIG. 6).

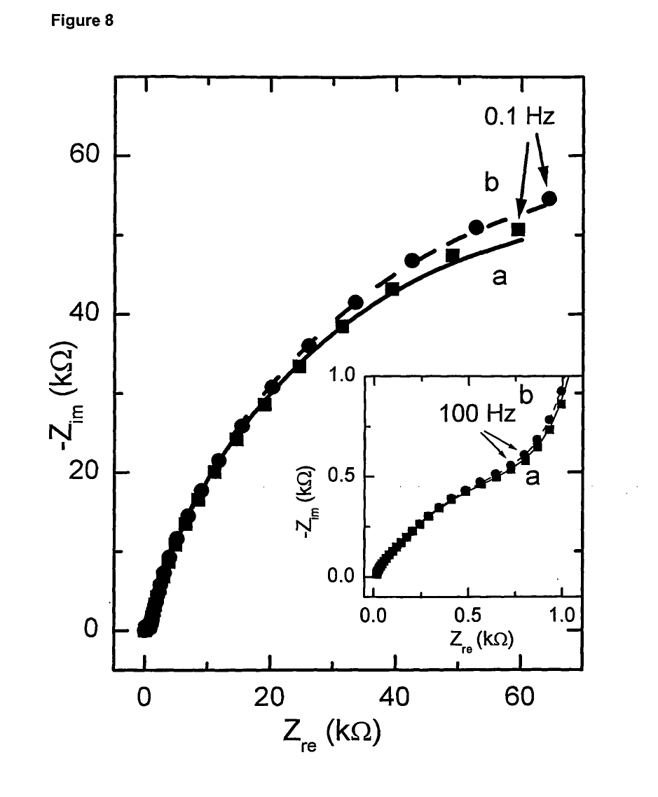

For many uncharged monolayers, different redox. probes may give qualitatively similar results, presumably because the interaction between the probe and the monolayer is not electrostatic (Boubour and Lennox, 2000; Finklea, 1996; Finklea et al., 1993). DNA, however, is negatively-charged and therefore, positively-charged probes such as Ru(NH₃)₆^{3+/2+} may enter the monolayer whereas negatively-charged probes such as Fe(CN)₆^{3-/4-} may not. These differences are for example reflected in the results shown in the examples herein, where R_{ct} with Ru(NH₃)₆^{3+/4+} is about 1 kΩ (FIG. 8), similar to that of a bare electrode, whereas with Fe(CN)₆^{3+/4+} and B-DNA the corresponding value is nearly 20 kΩ. Therefore, Ru(NH₃)₆^{3+/4+} is not a suitable probe for DNA since the charge transfer can essentially by-pass the monolayer.

The results disclosed in the examples herein illustrate that that under certain conditions, M-DNA may be a better conductor than B-DNA since both R_{ct} and Rₓ are smaller for M-DNA. In the examples, the difference between R_{ct} for B- and M-DNA tends to increase with increasing length whereas the difference in Rₓ decreases with increasing length of the DNA duplex. In the examples, the DNA was not directly attached to the electrode so that Rₓ and R_{ct} both contain terms in series for electron transfer from the DNA through the linker to the electrode. In alternative embodiments, the DNA may be attached directly or with linkers of variable lengths to resolve the influence of the linker. In some embodiments, the interconversion of B- and M-DNA may provide systems wherein both Rx and Rct can be modulated with changes in metal ion or pH.

### EXAMPLE NO. 1

In an example of some aspects of the invention, described in more detail below, monolayers of thiol-labelled DNA duplexes of 15, 20, and 30 base pairs were assembled on gold electrodes. Electron transfer was investigated by electrochemical impedance spectroscopy with Fe(CN)₆^{3-/4-} as a redox probe. The spectra, in the form of Nyquist plots, were analysed with a modified Randle circuit which included an additional component in parallel, Rₓ, for the resistance through the DNA. For native B-DNA Rₓ and R_{ct}, the charge transfer resistance, both increase with increasing length. M-DNA was formed by the addition of Zn²⁺ at pH 8.6 and gave rise to characteristic changes in the Nyquist plots which were not observed upon addition of Mg²⁺ or at pH 7.0. Rₓ and R_{ct} also increased with increasing duplex length for M-DNA but both were significantly lower compared to B-DNA. Therefore, certain metal ions can modulate the electrochemical properties of DNA monolayers and electron transfer via the metal DNA film is faster than that of the native DNA film.

Chemicals: Potassium hexaferricyanide, potassium hexaferrocyanide, hexaamineruthenium (III) chloride hexaammineruthenium (II) chloride, were from Aldrich and were ACS reagent grade. Zn(ClO₄)₂, Mg(ClO₄)₂ and Tris-ClO₄ were purchased from Fluka Co. The standard buffer was 20 mM Tris-ClO₄ at either pH 8.7 or 7.0. Other chemicals were analytical grade. All solutions were prepared in Millipore filtered water.

DNA: The probe DNAs were synthesized and purified with standard DNA synthesis methods at the Plant Biotechnology Institute, Saskatoon. The oligonuocleotides base sequences are: 15-mer DNA, 5′-AAC TAC TGG GCC ATC-(CH₂)₃—S—S—(CH₂)₃—OH-3′, target complementary sequence 5′-GAT GGC CCA GTA GTT-3′. 20mer DNA, 5′-AAC TAC TGG GCC ATC GTG AC-(CH₂)₃—S—S—(CH₂)₃—OH-3′, target complementary sequence 5′-GTC ACG ATG GCC CAG TAG TT-3′, 30mer DNA, 5′-GTG GCT AAC TAC GCA TTC CAC GAC CAA ATG-(CH₂)₃—S—S—(CH₂)₃—OH-3′, target complementary sequence 5′-CAT TTG GTC GTG GAA TGC GTA GTT AGC CAC-3′.

Electrode preparation: Gold disk electrodes (geometric surface area 0.02 cm²) and Ag/AgCl reference electrodes were purchased from Bioanalytical Systems. Before use, the electrodes were carefully polished with a 0.05 μm alumina slurry and then cleaned in 0.1 M KOH solution for a few minutes and then wash in Millipore H₂O, twice. The electrodes were carefully investigated by microscopy to ensure that there were no obvious defects. Finally, electrochemical treatment was preformed in the cell described below, by cyclic scanning from potential −0.1 to +1.25 V vs. Ag/AgCl in 0.5M H₂SO₄ solution until a stable gold oxidation peak at 1.1 V vs. Ag/AgCl was obtained (Finklea, 1996).

Preparation of DNA modified gold electrodes: DNA duplexes were prepared by adding 10 nmol of the disulphide-labeled DNA strands to 10 nmol of the complementary strands in 50 μl of 20 mM Tris-ClO₄ buffer pH 8.7 with 20 mM NaClO₄ for 2 hr at 20° C. The final double-stranded DNA concentration is about 100 μM. The freshly prepared gold electrodes were incubated with the DNA duplexes for 5 days in a sealed container. The electrodes were rinsed thoroughly with buffer solution (20 mM Tris-ClO₄ and 20 mM NaClO₄) and mounted into an electrochemical cell. B-DNA was converted to M-DNA by the addition of 0.4 mM ZnClO₄ for 2 hrs at pH 8.7.

X-Ray photoelectron Spectroscopy A Leybold MAX200 photoelectron spectrometer equipped with an Al-Kα radiation source (1486.6 eV) was used to collect photoemmission spectra. The base pressure during measurements was maintained less than 10⁻⁹ mbar in the analysis chamber. The take-off angle was 60°. The routine instrument calibration standard was the Au 4f_{7/2} peak (binding energy 84.0 eV).

Electrochemistry: A conventional three-electrode cell was used. All experiments were conducted at room temperature. The cell was enclosed in a grounded Faraday cage. The reference electrode was always isolated from the cell by a Luggin capillary containing the electrolyte. The salt-bridge reference electrode was used because of limiting Cl⁻ ion leakage for the normal Ag/AgCl reference electrode to the measurement system. The counter electrode was a platinum wire. Impedance spectroscopy was measured with a 1025 frequency response analyzer (FRA) interfaced to an EG&G 283 potentiostat/galvanostat via GPIB on a PC running Power Suite (Princeton Applied Research). Impedance was measured at the potential of 250 mV vs. Ag/AgCl, and was superimposed on a sinusoidal potential modulation of ±5 mV. The frequencies used for impedance measurements can range from 100 kHz to 100 mHz. The impedance data for the bare gold electrode, B-DNA and M-DNA modified gold electrode were analyzed using the ZSimpWin software (Princeton Applied Research). In all impedance spectra, symbols represent the experimental raw data, and the solid lines are the fitted curves.

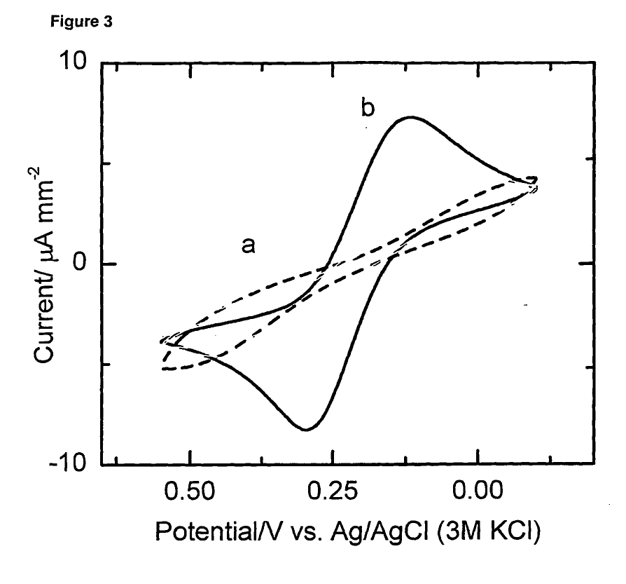

Assembly of the monolayer: Native duplex B-DNA was assembled on the gold surface as described in Materials and Methods. The monolayer was characterized by cyclic voltammetery with 4 mM K₃[Fe(CN)₆]/K₄[Fe(CN)₆] (1:1) mixture, as a redox probe. A typical scan is shown in FIG. 3; the bare gold electrode shows a characteristic quasi-reversible redox cycle with a peak separation of 158 mV. For the 20 base pair duplex assembled on the electrode, the peak current drops by over 95% and the separation between the oxidation and reduction peaks is increased indicating the presence of the DNA on the electrode and a reduced ability for electron transfer between the solution and the surface.

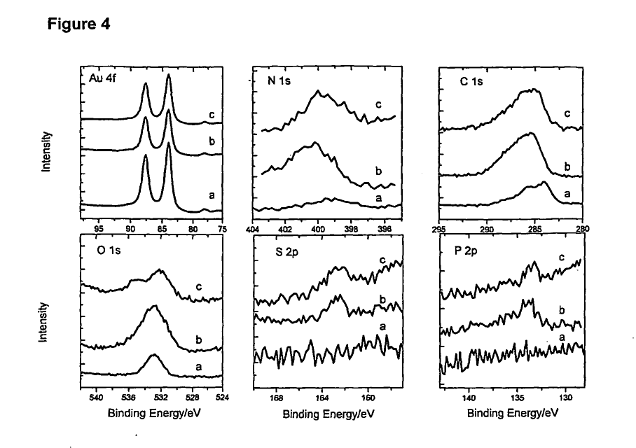

The gold surface was also analysed by X-ray photoelectron spectroscopy (XPS). As shown in FIG. 4, the intensity of the Au_{4f} peaks decreases upon attachment of the DNA (either B- or M-DNA) as expected for a modified surface (Kondo et al., 1998; Ishida et al., 1999). The S₂ₚ (162.4 eV), P₂ₚ (133 eV) and N₁ₛ (400 eV) peaks are evident in the spectra of B- and M-DNA but are not present in the spectrum of the bare gold providing good evidence for the attachment of a disulphide-linked DNA to the surface. Of particular interest is the observation that that the N₁ and O₁ₛ spectra for B- and M-DNA (after addition of Zn²⁺ at pH 8.7) are different. This is consistent with the zinc ions interacting with the DNA double helix and more specifically with the nitrogen and oxygen atoms of the base pairs (Lee et al., 1993; Aich et al., 1999).

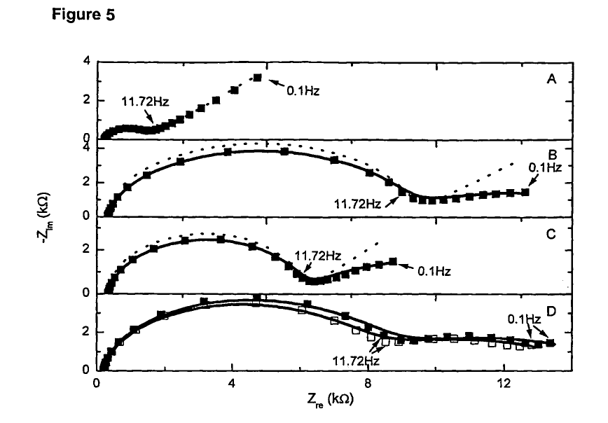

Impedance spectroscopy for B-DNA Impedance measurements were performed in the presence of 4 mM K₃[Fe(CN)₆]/K₄[Fe(CN)₆] (1:1) mixture, as the redox probe. FIG. 5A shows a Nyquist plot for the bare gold electrode which can be described as a semicircle near the origin at high frequencies followed by a linear tail with a slope of unity. Others have described similar curves and the data can be fit adequately by the Randles circuit of FIG. 1. The diameter of the semicircle is a measure of the charge transfer resistance, R_{ct}. For the 20-mer B-DNA (FIG. 5B) R_{ct} increases considerably compared to the bare electrode since electron transfer to the electrode is reduced. However, the low frequency region is no longer linear and cannot be fit adequately by a simple Randles circuit. Impedance measurements of B-DNA (and M-DNA, see below) in the absence of a redox probe (FIG. 6) demonstrated non-linear behavior which is not expected for a simple insulator. However, the curves of FIG. 6 could be fit with a simple circuit consisting of a capacitor with a resistance in parallel. This result suggests the presence of an additional interfacial resistance, Rₓ which can be added in parallel to the Randles circuit (FIG. 1). As shown in FIG. 5B, the modified circuit gives an excellent fit to the experimental data in both the low and high frequency zones.

Formation of M-DNA: Upon addition of Zn²⁺ to the 20-mer B-DNA modified gold electrode at pH 8.7 to form M-DNA, the impedance spectrum changed in a distinctive pattern with a reduction in Zᵢₘ and Zᵣₑ at both high and low frequencies (FIG. 5C). Control experiments demonstrated that M-DNA formation was complete within 2 hours. Again only the modified Randles circuit gives a good fit to the experimental data. Also shown in FIG. 5D are impedance spectra for the DNA modified electrode in a pH 7.0 buffer with and without Zn²⁺ and at pH 8.7 with Mg²⁺. Under these conditions, M-DNA does not form and only small changes in the impedance spectra are observed. The calculated values for Rₛ, Rₓ, R_{ct}, C and W are listed in Table 1. It is clear that there are significant decreases in Rₓ and R_{ct} upon formation of M-DNA which are not found upon addition of Mg²⁺ nor upon addition of Zn²⁺ at pH 7.0.

TABLE 1
Table I. DNA with pH and ion type^{a}

| | Bare | B-DNA | M-DNA | B-DNA | DNA with | DNA with |
|---|---|---|---|---|---|---|
| Element | Gold | pH 8.6 | pH 8.6 | PH 7.0 | Zn pH 7.0 | Mg pH 8.6 |
| Rs/Ω | 302 | 320 | 338 | 327 | 313 | 334 |
| Rx/Ω | | 16160 | 12850 | 15560 | 14650 | 14880 |
| C /μF | 2.6 | 0.288 | 0.285 | 0.289 | 0.318 | 0.355 |
| RCT/Ω | 1229 | 18830 | 10009 | 16180 | 15010 | 15360 |
| W /10- | 27 | 3.9 | 8.2 | 1.9 | 1.8 | 2.0 |
| 5Ωs-1/2 | | | | | | |

^{a}Values derived from the modified Randles circuit except for the bare Au electrode for which the data were fit to the unmodified Randles circuit.

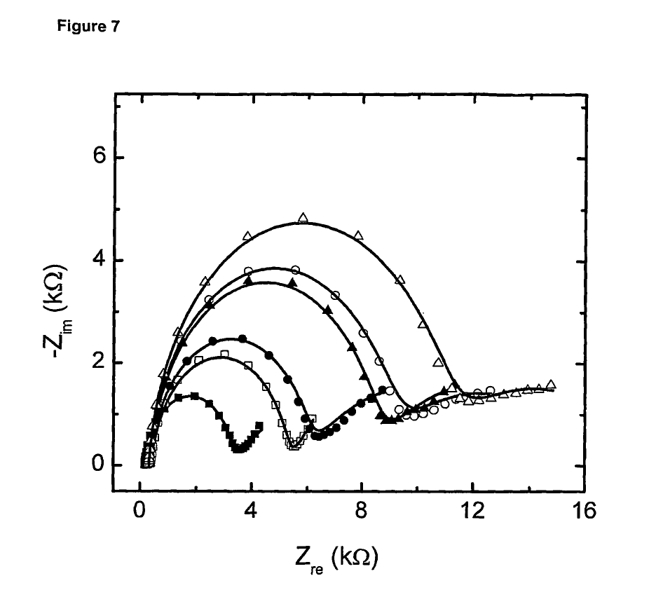

DNA sequence length: In order to provide further information concerning the elements in the suggested model, DNA duplexes of 15, 20, and 30 base pairs were used to modify the surface of the gold electrode. As shown in FIG. 7, all of the impedance spectra have the same characteristic shape and a fit to the modified Randles circuit is excellent. The calculated values for Rₛ, Rₓ, R_{ct}, C and W are listed in Table 2. There are two distinct trends. First, Rₓ and R_{ct} increase with increasing length for both B- and M-DNA. Second, for any length of duplex Rₓ and R_{ct} for M-DNA is less than the corresponding value for B-DNA. W, the Warburg impedance which represents mass transfer to the electrode is more variable but in all cases is higher for the M-DNA duplexes. As expected Rₛ, the solution resistance, is independent of duplex length and C, the double layer capacitance decreases with increasing length of the duplex.

TABLE 2
DNA with different length sequences

| | 15 mer | 15 mer | 20 mer | 20 mer | 30 mer | 30 mer |
|---|---|---|---|---|---|---|
| Element | B-DNA | M-DNA | B-DNA | M-DNA | B-DNA | M-DNA |
| Rs/Ω | 322 | 334 | 320 | 338 | 319 | 330 |
| Rx/Ω | 12500 | 7681 | 16160 | 12850 | 17630 | 15760 |
| C /μF | 0.679 | 0.621 | 0.288 | 0.285 | 0.291 | 0.271 |
| RCT/Ω | 7936 | 5326 | 18830 | 10009 | 26370 | 16720 |
| W /10- | 2.7 | 3.2 | 3.9 | 8.2 | 2.3 | 5.5 |
| 5Ωs-1/2 | | | | | | |

Ru(NH₃)₆^{3+/2+} redox probe: The redox probe in the above experiments was Fe(CN)₆^{3-/4-} which is negatively-charged and, therefore, will be repelled by the phosphodiester backbone of the DNA. Ru(NH₃)₆^{3+/2+}, on the other hand, is expected to be able to penetrate the monolayer. Impedance spectroscopy was performed with Ru(NH₃)₆^{3+/2+} as a redox probe for the 20 base pair B- and M-DNA duplexes (FIG. 8). As shown in the inset, R_{ct} is now very small and there is very little difference between the spectra for B-DNA and M-DNA.

### EXAMPLE NO. 2

In the previous example, the impedance spectroscopy of self-assembled monolayers (SAMS) of B-DNA and M-DNA is described, and it is shown that each gave characteristic values of resistance (R) and capacitance (C) which were dependent on DNA length and metal ion concentration. This example illustrates that single base pair mismatches in the DNA also give rise to well-defined changes in the impedance spectra so that a mismatch can be reliably distinguished from a perfect duplex under certain conditions.

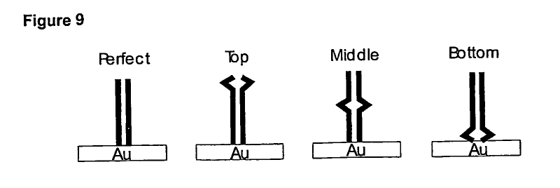

The DNA sequences and position of the mismatches are shown in FIG. 9, and in Table 3.

TABLE 3

| | | |
|---|---|---|
| Perfect | C3-SS-C3- | 5′-GTC ACG ATG GCC CAG TAG TT-3′ |
| match | | 5′-AAC TAC TGG GCC ATC GTG AC-3′ |
| DNA | | |
| Middle | C3-SS-C3- | 5′-GTC ACG ATG GCC CAG TAG TT-3′ |
| Mismatch | | 5′-AAC TAC TGG GTC ATC GTG AC-3′ |
| Top | C3-SS-C3- | 5′-GTC ACG ATG GCC CAG TAG TT-3′ |
| mismatch | | 5′-ATC TAC TGG GCC ATC GTG AC-3′ |
| Bottom | C3-SS-C3- | 5′-GTC ACG ATG GCC CAG TAG TT-3′ |
| Mismatch | | 5′-AAC TAC TGG GCC ATC GTG CC-3′ |

Methods used in this example are as set out in Example No. 1, unless indicated otherwise.

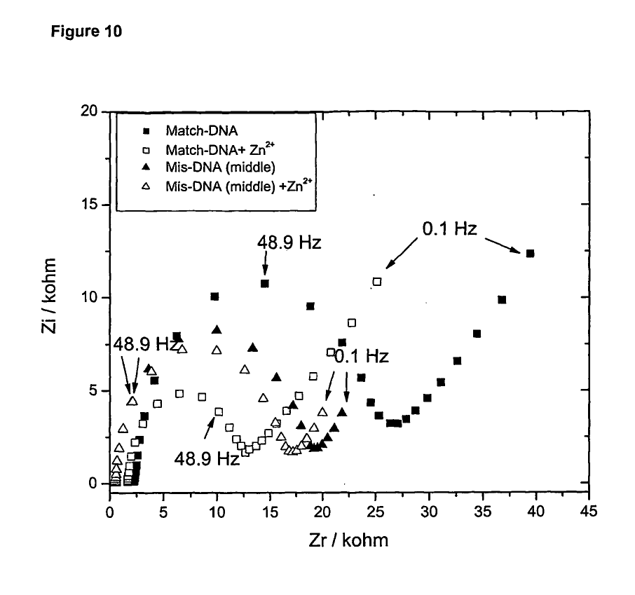

Impedance spectra for a perfect duplex and one containing a middle mismatch under B-DNA and M-DNA conditions are shown in the FIG. 10. Each point represents a value for Zᵢ and Zᵣ measured at a particular AC frequency. The points at 0.1 Hz and 49 Hz are for example highlighted and it can be seen that the corresponding values of Zᵢ and Zᵣ are very different for a perfect duplex and a mismatch and for B-DNA and M-DNA.

From the impedance spectra as shown in FIG. 10, it is possible to calculate the values of R and C with precision (as described in Example No. 1) and to use these to distinguish between a perfect duplex and a mismatch.

In some embodiments, different electrodes may give different values of R and C. Accordingly, in some embodiments, mismatch detection may be carried out using a matched set of electrodes. In alternative embodiments, because the difference between Z values for B-DNA and M-DNA may be more consistent and less dependent on the electrode and the experimental conditions, Z values may be measured at two frequencies for both B-DNA and M-DNA. From such data, it is possible to distinguish between a perfect duplex and a mismatched duplex. For example, ΔLᵢ may be defined as the difference between Zᵢ for B-DNA and M-DNA measured at low frequency (0.1 Hz) and ΔHᵣ may be defined as the difference between Zᵣ for B-DNA and M-DNA at high frequency (49 Hz). A Y factor may be defined as Y=ΔLᵢ×ΔLᵣ×ΔHᵢ×ΔHᵣ. In some embodiments, the measured Y factor for a perfect duplex may for example be about 1000 and for a mismatch may be from about 1 to about 40.

In one embodiment, a device that may for example be used for measuring Y factors is provided. Such a device comprising an array of electrodes each one of which would be individually addressable. A probe, such as a 20-mer duplex probe may be attached by a thiolate linkage to each electrode and the duplex denatured to leave only an attached single-stranded probe. This procedure may provide a more consistent electrode surface compared to attaching a single-strand directly. The target nucleic acid may then be hybridized to the electrodes and impedance measurements taken at two frequencies. The electrodes may then be treated to allow conversion to M-DNA, for example by treating with 0.2 mM ZnClO₄, and the impedance measurements repeated. In such embodiments, a measured Y factor below about 100 may be taken as indicative of a mismatch; whereas a value above about 100 may be taken to indicate a perfect duplex.

In some embodiments, careful measurements may allow the position of the mismatch to be detected, localizing the mismatch for example to the top, middle or bottom of the duplex. In some embodiments, such as single nucleotide polymorphism (SNP) detection, a sample from a heterozygote may for example give an intermediate Y value.

In some embodiments, polycrystalline gold electrodes may be used. Alternatively, monocrystalline electrodes may be used, which may improve the discrimination and enhance the sensitivity of the system.

In alternative embodiments, it will be appreciated that the systems of the invention may be used as data storage and readout devices in which information is stored in the form of the molecular configuration of a nucleic acid on an electrode.

### EXAMPLE NO. 3

This example illustrates electrochemical detection of a single-nucleotide polymorphism (SNP) using a DNA binding protein, the mismatch binding protein MutS. The results show that protein binding enhances the electrochemical discrimination between match and mismatch ds-DNA strands, facilitating the detection.

To demonstrate this aspect of the invention, electrochemical impedance spectroscopy (EIS) was used to analyze two types of gold modified electrodes. A first electrode was coated with a ds-DNA monolayer that was entirely complementary in its sequence of 20 base pairs (I: 5′ MC TAC TGG GCC ATC GTG AC 3′-(CH3)3-SH; II: 5′GTC ACG ATG GCC CAG TAG TT-3′). A second electrode was coated with a monolayer of ds-DNA that contained a one-base-pair-mismatch in the second from-top position of the strand ((I: 5′ MC TAC TGG GCC ATC GTG AC 3′-(CH3)3-SH; C-II: 5′GTC ACG ATG GCC CAG TAG CT-3′).

In order to optimize protein binding to the DNA monolayer, dilute monolayers of DNA were constructed. The DNA monolayer was diluted by reaction of the DNA-coated electrode with butanethiol to produce a surface comprised predominantly of butanethiol, having isolated molecules of ds-DNA. In alternative embodiments, other diluents may be used to lower the density of a nucleic acid monolayer on an electrode, such as: alkane thiols; alkylthiols; arylthiols; alkyl and arylthioethers; anilines and pyridine derivatives (or other nitorgen-containing base, such as an alkylamine or arylamines); selenolates or selenols; and, particularly for other metal surfaces, such as silver surfaces, diluents may be alkyl alcohols.

This example shows that this “dilute ds-DNA” surface on the electrode is amenable to electrochemical analysis, particularly for assays that involve protein binding to the nucleic acid. An aspect of the invention is therefore a method of titrating the density of nucleic acids in a monolayer on an electrode, using a diluent under conditions whereby the diluent moiety replaces nucleic acids on the surface of the electrode.

To prepare a protein for coupling to an electrode, it may be necessary to purify the protein. Purification may for example be carried out to remove from the protein preparation contaminating thiols or disulfides. In this example, MutS was purified by dialyzing a commercial MutS preparation, and concentrating the protein into a binding buffer. In alternative embodiments, a wide variety of DNA binding proteins may be utilized, such as native and recombinant sequence specific DNA binding proteins. For example, alternative DNA binding proteins may include sequence specific or non-sequence-specific DNA binding proteins selected from the group consisting of: DNA binding enzymes such as deoxyribonucleases, DNA or RNA polymerases, topoisomerases, DNA methylases, restriction endonucleases, DNA repair enzymes; non-enzymatic DNA binding proteins, such as histones, Hu proteins, HMG proteins, transcription factors, repressors, and activators. Novel sequence specific DNA binding proteins may for example be prepared for use in aspects of the invention (see for example Isalan et al., 2001). In alternative embodiments, the nucleic acid binding moiety may be non-proteinaceous, such as DNA binding drugs. In alternative embodiments, the nucleic acid may for example be an RNA, RNA duplex or RNA/DNA duplex.

In the present example, the binding conditions used were as follows. The concentration of the purified MutS (detected by UV spectroscopy at 280 nm) preparation was 8-20 μg/μl. The binding reaction was carried out for 20 minutes at room temperature. The addition of 5 mM MgClO2 was found to be beneficial for MutS binding. After binding, the surface was washed with buffer and dried under N₂ gas.

The dilution of the monolayer was carried out by incubation of the DNA monolayer coated electrode in 1 mM butanethiol containing 100 mM Tris-buffer, pH 7.0, for 20 minutes.

Faradaic impedence measurements were made in the presence of 5 mM [Fe(CN)₆]^{3-/4-} in 20 mM Tris-ClO₄ buffer (pH 8.5) containing 100 mM NaClO₄.

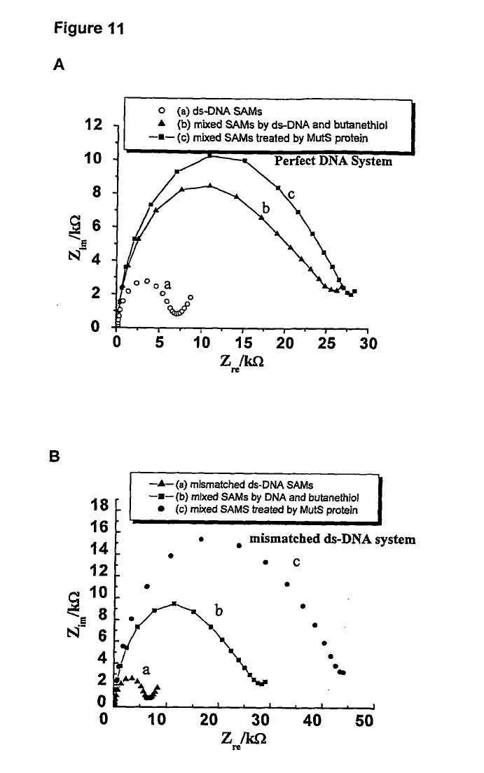

The dilution of the densely packed ds-DNA monolayer with butanethiol resulted in an increase in the impedance of the monolayer for both complementary and mismatched-DNA strands. After incubating the diluted DNA monolayer in MutS, under conditions as described above, the impedance increased further for both complementary and mismatched stands. However, for the electrode coated with the DNA having a mismatch, there is a remarkable increase in the impedance signal compared to the increase observed for the complementary DNA coated electrode (as shown in FIG. 11).

TABLE 3

| | | | | | ΔLᵣ = (Z_{r,L MutS}, −Z_{r,L,B-DNA}) ₁₁ | | | |
|---|---|---|---|---|---|---|---|---|
| | | | | | ΔLᵢ = (Z_{i,L MutS}, −Z_{i,L,B-DNA});₁₁ | | | |
| | Low Frequency | | High Frequency | | ΔHᵣ = (Z_{r,H MutS}, −Z_{r,H, B-DNA}); | | | |
| | (1 Hz) | | (11.7 Hz) | | ΔHᵢ = (Z_{i,H,MutS}, −Z_{r,H, B-DNA}). | | | |
| Samples | Z_{r,L} | Z_{i,L} | Z_{r,H} | Z_{i,H} | ΔLᵣ | ΔLᵢ | ΔHᵣ | ΔHᵢ |
| Perfect ds-DNA | 26.88 | 2.51 | 13.91 | 7.84 | 1.50 | −0.27 | 1.10 | 2.15 |
| Perfect ds-DNA + | 28.38 | 2.24 | 15.01 | 9.99 | | | | |
| MutS Protein | | | | | | | | |
| Mismatched ds- | 28.68 | 2.87 | 14.78 | 9.01 | 14.36 | 0.40 | 1.52 | 5.39 |
| DNA | | | | | | | | |
| Mismatched ds- | 44.04 | 3.27 | 16.30 | 15.4 | | | | |
| DNA + MutS | | | | | | | | |
| Protein | | | | | | | | |

| | | | Y₁ factor | | Y₂ factor | Y₃ factor |
|---|---|---|---|---|---|---|
| | | | | | | ΔLᵢ x |
| | ΔLᵣ x ΔHᵣ | ΔLᵢ x ΔHᵢ | ΔLᵣ x ΔHᵢ | ΔLᵢ x ΔHᵣ | ΔLᵢ x ΔHᵢ x ΔHᵢ | ΔHᵢ x ΔLᵢ x ΔHᵢ |
| Perfect | 1.65 | 0.58 | 3.23 | 0.29 | 3.55 | 0.96 |
| DNA system | | | | | | |
| Mismatched | 21.82 | 2.16 | 78.40 | 0.61 | 117.65 | 47.06 |
| DNA system | | | | | | |

The Y factor = ΔLr x ΔHi

In alternative embodiments, other nucleic acid binding moieties may be used in methods of the invention. For example, alternative DNA binding proteins may include sequence specific or non-sequence-specific DNA binding proteins selected from the group consisting of: DNA binding enzymes such as deoxyribonucleases, DNA or RNA polymerases, topoisomerases, DNA methylases, restriction endonucleases, DNA repair enzymes; non-enzymatic DNA binding proteins, such as histones, Hu proteins, HMG proteins, transcription factors, repressors, and activators. Novel sequence specific DNA binding proteins may for example be prepared for use in aspects of the invention (see for example Isalan et al., 2001). In alternative embodiments, the nucleic acid binding moiety may be non-proteinaceous, such as DNA binding drugs. In alternative embodiments, the nucleic acid may for example be an RNA, RNA duplex or RNA/DNA duplex.

### REFERENCES

The following documents are incorporated herein by reference:

Aich, P., L. Labiuk, W. Tari, L. J. T. Delbaere, W. J. Roesler, Falk K. J., R. P. Steer, and J. S. Lee. 1999. M-DNA: A complex between divalent metal ions and DNA which behaves as a molecular wire. J. Mol. Biol. 294:477-483.

Aich, P., R. J. S. Skinner, S. D. Wettig, R. P. Steer, and J. S. Lee. 2002. Long range molecular wire behaviour in a metal complex of DNA. J. Biomol. Struct. & Dyn. 20:1-6.

Ambroise, A. and B. G. Maiya. 2000. Ruthenium(II) complexes of redox-related, modified dipyridophenazine ligands: Synthesis, characterization, and DNA interaction. Inorg. Chem. 39:4256-4263.

Bard, A. J. and L. R. Faulkner. 2001. Electrochemical Methods: fundamentals and applications. John Wiley & Sons, Inc., New York.

Bixon, M., B. Giese, S. Wessely, T. Langenbacher, M. E. Michel-Beyerle, and J. Jortner. 1999. Long-range charge hopping in DNA. Proc. Nat. Acad. Sci. USA 96:11713-11716.

Boon, E. M., D. M. Ceres, T. G. Drummond, M. G. Hill, and J. K. Barton. 2000. Mutation detection by electrocatalysis at DNA-modified electrodes. Nat. Biotechnol. 18:1096-1100.

Boubour, E. and R. B. Lennox. 2000. Insulating Properties of Self-Assembled Monolayers Monitored by Impedance Spectroscopy. Langmuir 16:4222-4228.

Braun, E., Y. Eichen, U. Sivan, and G. Ben Yoseph. 1998. DNA-templated assembly and electrode attachment of a conducting silver wire. Nature 391:775-778.

Carter, M. T. and A. J. Bard. 1987. Voltammetric studies of the interaction of tris(1,10-phenanthroline)cobalt(III) with DNA. J. Am. Chem. Soc. 109:7528-7530.

Finklea, H. O., D. A. Snider, J. Fedyk, E. Sabatani, Y. Gafni, and I. Rubinstein. 1993. Characterization of octadecanethiol-coated gold electrodes as microarray electrodes by cyclic voltammetry and ac impedance spectroscopy. Langmuir 9:3660-3667.

Finklea, H. O. 1996. Electroanalytical Chemistry. Marcel Dekker Inc., New York.

Fodor, S. P. A., R. P. Rava, X. H. C. Huang, A. C. Pease, and C. P. Holmes. 1993. Multiplexed biochemical assays with biological chips. Nature 364:555-556.

Hashimoto, K., K. Ito, and Y. Ishimori. 1994. Sequence-specific gene detection with a gold electrode modified with DNA probes and an electrochemically active dye. Anal. Chem. 66:3830-3833.

Heme, T. M. and M. J. Tarlov. 1997. Characterization of DNA probes immobilized on gold surface. J. Am. Chem. Soc. 119:8916-8920.

Ishida, T., N. Choi, W. Mizutani, H. Tokumoto, I. Kojima, H. Azehara, H. Hokari, U. Akiba, and M. Fujihira. 1999. High-resolution X-ray photoelectron spectra of organosulfur monolayers on Au(111): S(2p) spectral dependence on molecular species. Langmuir 15:6799-6806.

Isalan M., Klug A., Choo Y. 2001. A rapid, generally applicable method to engineer zinc fingers illustrated by targeting the HIV-1 promoter. Nat Biotechnol. July;19(7):656-60.

Janek, R. P., W. R. Fawcett, and A. Ulman. 1998. Impedance spectroscopy of self-assembled monolayers on Au(111): sodium ferrocyanide charge transfer at modified electrodes. Langmuir 14:3011-3018.

Kelley, S. O., J. K. Barton, N. M. Jackson, and M. G. Hill. 1997. Electrochemistry of methylene blue bound to a DNA-modified electrode. Bioconjugate Chem. 8:31-37.

Kelly, S. O., N. M. Jackson, M. G. Hill, and J. K. Barton. 1999. Long-range electron transfer through DNA films. Angew. Chem. Int. Ed. 38:941-945.

Kertesz, V., N. A. Whittemore, J. Q. Chambers, M. S. McKinney, and D. C. Baker. 2000. Surface titration of DNA-modified gold electrodes with a thiol-tethered anthraquinone. J. Electroanal. Chem. 493:28-36.

Kharitonov, A. B., L. Alfonta, E. Katz, and I. Willner. 2000. Probing of bioaffinity interactions at interfaces using impedance spectroscopy and chronopotentiometry. J. Electroanal. Chem. 487:133-141.

Kondo, T., M. Yanagida, K. Shimazu, and K. Uosaki. 1998. Determination of thickness of a self-assembled monolayer of dodecanethiol on Au(111) by Angle-resolved X-ray photoelectron spectroscopy. Langmuir 14:5656-5658.

Lee, J. S., L. J. P. Latimer, and R. S. Reid. 1993. A coopoerative conformational change in duplex DNA induced by Zn²⁺ and other divalent metal ions. Biochem. Cell Biol. 71:162-168.

Macdonald, J. R. 1987. Impedance Spectroscopy. John Wiley & Sons, New York.

Millan, K. M., A. Saraullo, and S. R. Mikkelsen. 1994. Diagnosis of cystic fibrosis in PCR-amplified human DNA using voltammetric DNA sensors. Anal. Chem. 66:2943-2948.

Murphy, C. J., Arkin M. R., Y. Enkins, N. D. Ghatlia, Bossmann S. H., Turro N. J., and J. K. Barton. 1993. Long-range photoinduced electron-transfer through a DNA helix. Science 262:1025-1029.

Patolsky, F., A. Lichtenstein, and I. Willner. 2001. Electronic transduction of DNA sensing processes on surfaces: Amplification of DNA detection and analysis of single-base mismatches by tagged liposomes. J. Am. Chem. Soc. 123:5194-5205.

Porath, D., A. Bezryadin, S. de Vries, and C. Dekker. 2000. Direct measurement of electrical transport through DNA molecules. Nature 403:635-638.

Pressprich, K. A., S. G. Maybury, R. E. Thomas, R. W. Linton, E. A. Irene, and R. W. Murray. 1989. Molecular sieving by electropolymerized porphyrin films only a few monolayers thick. J. Phys. Chem. 93:5568-5574.

Rakitin, A., P. Aich, C. Papaopoulos, Y. Kobzar, A. S. Vedeneev, J. S. Lee, and J. M. Xu. 2001. Metallic conduction through engineered DNA: DNA nanoelectronic building blocks. Phys. Rev. Lett. 86:3670-3673.

Randles, J. E. B. 1947. Kinetics of rapid electrode reactions. Discussions Faraday Soc. 1:11-19.

Schena, M., D. Shalon, R. Heller, A. Chai, P. O. Brown, and R. W. Davis. 1996. Parallel human genome analysis: Microarray-based expression monitoring of 1000 genes. Proc. Nat. Acad. Sci. USA 93:10614-10619.

Storm, A. J., J. van Noort, S. de Vries, and C. Dekker. 2001. Insulating behavior for DNA molecules between nanoelectrodes at the 100 nm length scale. Appl. Phys. Lett. 79:3881-3883.

Takagi, M. 2001. Threading intercalation to double-stranded DNA and the application to DNA sensing. Electrochemistry array technique. Pure Appl. Chem. 73:1573-1577.

Tani, A., A. J. Thomson, and J. N. Butt. 2001. Methylene blue as an electrochemical discriminator of single- and double-stranded oligonucleotides immobilised on gold substrates. Analyst 126:1756-1759.

Ulman, A. 1996. Formation and structure of self-assembled monolayers. Chem. Rev. 96:1533-1554.

Yan, F. and O. A. Sadik. 2001a. Enzyme-modulated cleavage of dsDNA for studying interfacial biomolecular interactions. J. Am. Chem. Soc. 123:11335-11340.

Yan, F. and O. A. Sadik. 2001b. Enzyme-modulated cleavage of dsDNA for supramolecular design of biosensors. Anal. Chem. 73:5272-5280.

### CONCLUSION

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. All publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A method for detecting binding of a nucleic acid binding moiety to a nucleic acid comprising measuring the impedance of a layer of the nucleic acid on an electrode by AC impedance spectroscopy in the presence of the moiety.

2. The method of claim 1, wherein the moiety is a protein.

3. The method of claim 1, wherein the nucleic acid is a DNA.

4. The method of claim 1, wherein the layer of the nucleic acid is diluted with a diluent.

5. The method of claim 1, further comprising diluting the layer of the nucleic acid with a diluent prior to the step of detecting, to reduce the concentration of the nucleic acid on the electrode.

6. The method of claim 1, wherein the nucleic acid is a nucleic acid duplex.

7. The method of claim 1, wherein the moiety is a protein and the protein is a mismatch binding protein.

8. The method of claim 1, wherein the nucleic acid is a nucleic acid duplex, and the nucleic acid duplex comprises a single nucleotide polymorphism, and the single nucleotide polymorphism is detectable by Faradaic impedence measurement.

9. A method for detecting binding of a nucleic acid binding moiety to a nucleic acid tethered to an electrode in an electrochemical circuit, comprising:
a) applying electrical energy to the electrode in the electrochemical circuit;
b) collecting electrochemical circuit data related to the impedance of the nucleic acid on the electrode in the circuit; and,
c) fitting the electrochemical circuit data to a circuit model to obtain circuit performance information indicative of binding of the nucleic acid binding moiety to the nucleic acid.

10. The method of claim 9, wherein collecting electrochemical circuit data comprises measuring impedance spectra.

11. The method of claim 10, wherein the impedance spectra are measured in the frequency domain.

12. The method of claim 9, wherein the electrochemical circuit data comprises a measure of complex impedance.

13. The method of claim 9, wherein the electrical energy is applied in an impedance spectroscopy system, and the impedance spectroscopy system comprises applying a sinusoidal signal at a constant frequency and a constant amplitude within a discrete period.

14. The method of claim 9, wherein the circuit model comprises as circuit elements:
a solution resistance Rs;
a charge transfer resistance RCT;
a constant-phase element CPE;
a mass transfer element W (Warburg impedance); and,
a resistance in parallel Rx;
and wherein the circuit elements are arranged as follows:

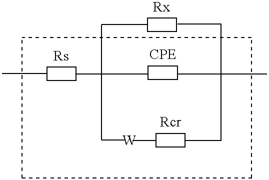

15. The method of claim 9, wherein the nucleic acid is a deoxyribonucleic acid duplex.

16. The method of claim 9, wherein the nucleic acid comprises a metal-containing nucleic acid duplex comprising a first strand of nucleic acid and a second strand of nucleic acid, the first and the second nucleic acid strands comprising a plurality of nitrogen-containing aromatic bases covalently linked by a backbone, the nitrogen-containing aromatic bases of the first nucleic acid strand being joined by hydrogen bonding to the nitrogen-containing aromatic bases of the second nucleic acid strand, the nitrogen-containing aromatic bases on the first and the second nucleic acid strands forming hydrogen-bonded base pairs in stacked arrangement along the length of the conductive metal-containing nucleic acid duplex, the hydrogen-bonded base pairs comprising an interchelated divalent metal cation coordinated to a nitrogen atom in one of the aromatic nitrogen-containing aromatic bases.

17. The method of claim 9, further comprising comparing the circuit performance information of a first nucleic acid duplex to the circuit performance information of a second nucleic acid duplex.

18. The method of claim 17, wherein the first nucleic acid duplex is B-DNA and the second nucleic acid duplex is a metal-containing nucleic acid duplex comprising a first strand of nucleic acid and a second strand of nucleic acid, the first and the second nucleic acid strands comprising a plurality of nitrogen-containing aromatic bases covalently linked by a backbone, the nitrogen-containing aromatic bases of the first nucleic acid strand being joined by hydrogen bonding to the nitrogen-containing aromatic bases of the second nucleic acid strand, the nitrogen-containing aromatic bases on the first and the second nucleic acid strands forming hydrogen-bonded base pairs in stacked arrangement along the length of the conductive metal-containing nucleic acid duplex, the hydrogen-bonded base pairs comprising an interchelated divalent metal cation coordinated to a nitrogen atom in one of the aromatic nitrogen-containing aromatic bases

19. The method of claim 9, wherein the circuit performance information is plotted on a Nyquist plot.

20. The method of claim 9, wherein a plurality of nucleic acids form a monolayer of nucleic acid duplexes on the electrode.

21. The method of claim 9, wherein the electrochemical circuit comprises an aqueous electrolyte and the nucleic acid is tethered and solvated in the aqueous electrolyte.

22. The method of claim 21, further comprising a redox probe in the aqueous solution.

23. The method of claim 9, wherein the nucleic acid duplex is an double helix.

24. A system for detecting binding of a nucleic acid binding moiety to a nucleic acid tethered to an electrode in an electrochemical circuit, the system comprising:
a) means for applying electrical energy to the electrode in the electrochemical circuit;
b) means for collecting electrochemical circuit data related to the impedance of the nucleic acid duplex on the electrode in the circuit; and,
c) means for fitting the electrochemical circuit data to a circuit model to obtain circuit performance information indicative of binding of the nucleic acid binding moiety to the nucleic acid.

25. A system for detecting binding of a nucleic acid binding moiety to a nucleic acid in a nucleic acid tethered to an electrode in an electrochemical circuit, the system comprising:
a) an electrical current source for applying electrical energy to the electrode in the electrochemical circuit;
b) a controller for collecting electrochemical circuit data related to the impedance of the nucleic acid duplex on the electrode in the circuit; and,
c) an analyzer for fitting the electrochemical circuit data to a circuit model to obtain circuit performance information indicative of binding of the nucleic acid binding moiety to the nucleic acid.

26. The system of claim 25, further comprising a display for displaying the circuit performance information.

27. The system of claim 25, further comprising a recorder for recording the circuit performance information.

28. A method for detecting binding of a nucleic acid binding moiety to a nucleic acid by measuring electrochemical circuit data related to the impedence of a nucleic acid layer on an electrode substantially as hereinbefore described and with reference to the examples and drawings.


SEQUENCE LISTING (13 sequences)

SEQ ID NO: 1 - DNA, 15 bp, Artificial
  Probe
aactactgggccatc

SEQ ID NO: 2 - DNA, 15 bp, Artificial
  Target
gatggcccagtagtt

SEQ ID NO: 3 - DNA, 20 bp, Artificial
  Probe
aactactgggccatcgtgac

SEQ ID NO: 4 - DNA, 20 bp, Artificial
  Target
gtcacgatggcccagtagtt

SEQ ID NO: 5 - DNA, 30 bp, Artificial
  Probe
gtggctaactacgcattccacgaccaaatg

SEQ ID NO: 6 - DNA, 30 bp, Artificial
  Target
catttggtcgtggaatgcgtagttagccac

SEQ ID NO: 7 - DNA, 20 bp, Artificial
  Probe
gtcacgatggcccagtagtt

SEQ ID NO: 8 - DNA, 20 bp, Artificial
  Target
aactactgggccatcgtgac

SEQ ID NO: 9 - DNA, 20 bp, Artificial
  Target
aactactgggtcatcgtgac

SEQ ID NO: 10 - DNA, 20 bp, Artificial
  Target
atctactgggccatcgtgac

SEQ ID NO: 11 - DNA, 20 bp, Artificial
  Target
aactactgggccatcgtgcc

SEQ ID NO: 12 - DNA, 20 bp, Artificial
  Probe
aactactgggccatcgtgac

SEQ ID NO: 13 - DNA, 20 bp, Artificial
  Target
gtcacgatggcccagtagct